(19) United States
(12) Patent Application Publication
(10) Pub. No.: US 2014/0171389 A1
(43) Pub. Date: Jun. 19, 2014

(54) METHODS FOR TREATING CANCER

(71) Applicant: Karen Curd, South San Francisco, CA (US)

(72) Inventors: Charles Hart, South San Francisco, CA (US); Mark Matteucci, South San Francisco, CA (US); John Curd, South San Francisco, CA (US)

(73) Assignee: THRESHOLD PHARMACEUTICALS, INC., South San Francisco, CA (US)

(21) Appl. No.: 14/009,068

(22) PCT Filed: Mar. 30, 2012
(86) PCT No.: PCT/US2012/031677

§ 371 Date: Dec. 16, 2013

Related U.S. Application Data
(60) Provisional application No. 61/470,921, filed on Apr. 1, 2011.

(51) Int. Cl. A61K 31/675; A61K 45/06

(52) U.S. Cl. CPC A61K 31/675; A61K 45/06
USPC 514/64; 514/94

(57) ABSTRACT

Administration of TH-302 or another hypoxia activated prodrug in combination with a pharmacological agent that down-regulates or inhibits homology directed repair (HDR) is useful for treating cancer.

### CROSS-REFERENCE TO RELATED APPLICATIONS

This application claims priority under 35 U.S.C. 119(e) of U.S. provisional application No. 61/470,921 filed on Apr. 1, 2011, the contents of which is incorporated herein by reference.

### FIELD OF INVENTION

The present invention relates to combination cancer treatment with hypoxia activated prodrugs and homology directed repair inhibitors.

### BACKGROUND OF THE INVENTION

TH-302 is a hypoxia activated prodrug in clinical development for the treatment of cancer. See PCT Publication Nos. 2007/002931; 2008/083101; 2010/048330; 2012/006032; and 2012/009288; and U.S. Patent Application No. 61/475,844, filed 15 Apr. 2011, each of which is incorporated herein by reference. TH-302 releases the DNA cross-linking bromo-isophosphoramidate mustard (Br-IPM) under hypoxic conditions. TH-302 is a hypoxia-activated prodrug (HAP) whose mechanism of action includes DNA alkylation, resulting in DNA cross-links. While there have been promising reports from clinical trials on the anti-cancer effectiveness of TH-302, both in monotherapy and in combination therapy with other anti-cancer agents, there remains a need for increasing the effectiveness of cancer treatment using TH-302 and other hypoxia activated prodrugs. The present invention meets this need.

### SUMMARY OF THE INVENTION

In one aspect, the present invention provides methods of treating cancer with TH-302 or another hypoxia activated prodrug with identical or similar mechanism of action, such methods comprising administering a therapeutically effective amount of TH-302 or another hypoxia activated prodrug in combination with a therapeutically effective amount of a drug, including an FDA or another regulatory authority approved drug, that down-regulates (or inhibits) homology directed repair (HDR) of DNA (which may be referred to herein as an “HDR inhibitor”).

In various embodiments, the hypoxia activated prodrug is a compound of Formula I:

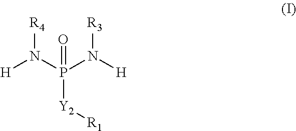

wherein Y₂ is O, S, NR₆, NCOR₆, or NSO₂R₆ wherein R₆ is C₁-C₆ alkyl, C₁-C₆ heteroalkyl, aryl, or heteroaryl; R₃ and R₄ are independently selected from the group consisting of 2-haloalkyl, 2-alkylsulfonyloxyalkyl, 2-heteroalkylsulfonyloxyalkyl, 2-arylsulfonyloxyalkyl, and 2-heteroalkylsulfonyloxyalkyl; R₁ has the formula L-Z₃; L is C(Z₁)₂; each Z₁ independently is hydrogen, halogen, C₁-C₆ alkyl, C₁-C₆heteroalkyl, aryl, heteroaryl, C₃-C₈ cycloalkyl, heterocyclyl, C₁-C₆ acyl, C₁-C₆ heteroacyl, aroyl, or heteroaroyl; or L is:

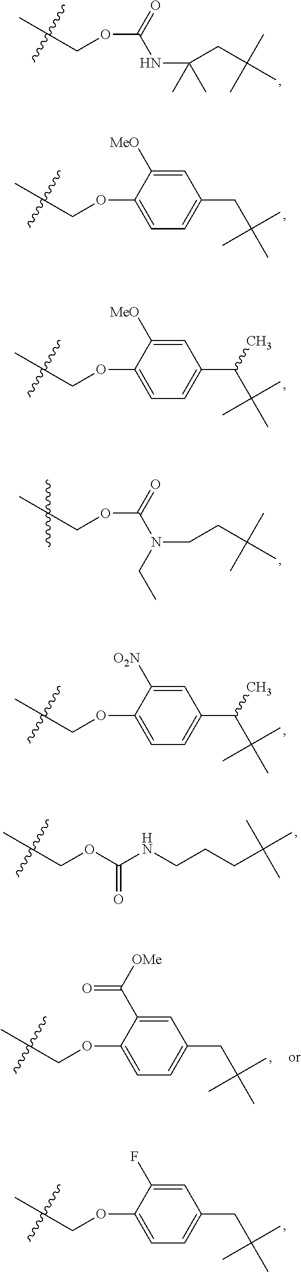

Z₃ is a bioreductive group having a formula selected from the group consisting of:

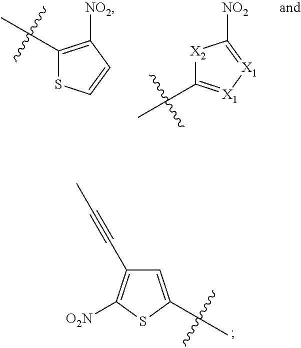

wherein each X₁ is independently N or CR₈; X₂ is NR₇, S, or O; each R₇ is independently C₁-C₆ alkyl, C₁-C₆ heteroalkyl, C₃-C₈ cycloalkyl, heterocyclyl, aryl or heteroaryl; and R₈ is independently hydrogen, halogen, cyano, CHF₂, CF₃, CO₂H, amino, C₁-C₆ alkyl, C₁-C₆ heteroalkyl, C₁-C₆ cycloalkyl, C₁-C₆ alkoxy, C₁-C₆ alkylamino, C₁-C₆ dialkylamino, aryl, CON(R₇)₂, C₁-C₆ acyl, C₁-C₆ heteroacyl, aroyl or heteroaroyl; or a pharmaceutically acceptable salt thereof. In various embodiments, the compound utilized in the methods of this invention is a compound of Formula I selected from the group consisting of TH-281, TH-302, and TH-308 (structures provided below).

In one embodiment, the hypoxia activated prodrug is TH-302. In various embodiments, the TH-302 is administered at a dose and frequency described in PCT Publication Nos. 2007/002931; 2008/083101; 2010/048330; 2012/006032; and 2012/009288; and U.S. Patent Application No. 61/475,844, filed 15 Apr. 2011, each of which is incorporated herein by reference. In some embodiments, the TH-302 is administered at a dose of 240 mg/m² or 340 mg/m² once per week, typically in four-week cycles, in which TH-302 is administered on days 1, 8, and 15.

In other embodiments, the hypoxia activated prodrug is PR104, AQ4N, tirapazamine, or CEN-209.

In various embodiments, the HDR inhibitor used in the methods of the invention is selected from the group consisting of proteosome inhibitors (including but not limited to bortezomib), histone deacetylase (HDAC) inhibitors (including but not limited to vorinostat, also known as SaHa), and tyrosine kinase inhibitors (including but not limited to imatinib, gefitinib, and erlotinib).

In various embodiments, the patient treated has been identified as having a cancer with a low level of HDR activity. As demonstrated according to this invention, the lower the HDR activity in the cell, the more susceptible the cancer is to treatment with TH-302 or other hypoxia activated prodrugs with similar mechanism of action, and the higher the HDR activity in the cell, the less susceptible the cancer is to treatment with TH-302 or the other hypoxia activated prodrug. Conversely, however, patients with high HDR activity levels will benefit from treatment in accordance with the methods of the invention, relative to a treatment comprising a hypoxia activated prodrug that excludes an HDR inhibitor. Thus, patients suitable for treatment in accordance with the methods of the invention include those having cancer with any detectable level of HDR activity. Any known method for assessing HDR activity is suitable for use according to the present methods.

In various embodiments, the present invention provides methods of treating cancer, such methods comprising administering a therapeutically effective amount of the compound of Formula I, in combination with a therapeutically effective amount of an HDR inhibitor, including but not limited to an HDR inhibitor selected from the group consisting of bortezomib, vorinostat, imatinib, gefitinib, or erlotinib. In certain embodiments, the compound of Formula I is TH-302. In various embodiments, an anti-cancer drug in addition to the hypoxia activated prodrug and the HDR inhibitor is administered. In some embodiments, this additional drug is docetaxel, doxorubicin, gemcitabine, or pemetrexed.

In certain embodiments, the cancer is a blood cancer such as a leukemia, a lymphoma, and the like, or is a gastrointestinal stromal tumor (GIST), a pancreatic cancer, a sarcoma, or a lung cancer. These and other cancers treated according to the present invention are described herein below. In some embodiments, the cancer is a solid tumor, i.e., the cancer is not a blood cancer such as leukemia and lymphoma.

In another aspect, the present invention provides pharmaceutically acceptable formulations and unit dose forms suitable for use in the methods of the present invention. In one embodiment, the hypoxia activated prodrug and the HDR inhibitor are formulated separately in distinct unit dose forms. In another embodiment, the hypoxia activated prodrug and HDR inhibitor are formulated together in an admixture or other combination pharmaceutical formulation and combination unit dose forms. In various embodiments, the hypoxia activated prodrug in the formulation and unit dose forms is TH-302. In some embodiments, the pharmaceutically acceptable formulations and unit dose forms further comprise at least one pharmaceutically acceptable excipient. Suitable pharmaceutically acceptable excipients are well known to the skilled artisan.

In certain embodiments, the pharmaceutically acceptable formulations and unit dose forms comprise a therapeutically effective amount of the compound of Formula I, e.g., TH-302. In certain other embodiments, the pharmaceutically acceptable formulations and unit dose forms further comprise a therapeutically effective amount of the HDR inhibitor.

### DETAILED DESCRIPTION OF THE INVENTION

The practice of the present technology includes the use of conventional techniques of molecular biology (including recombinant techniques), microbiology, cell biology, biochemistry and immunology, which are within the skill of the art.

### DEFINITION

In this specification and in the claims that follow, reference will be made to a number of terms that shall be defined to have the meanings below. All numerical designations, e.g., pH, temperature, time, concentration, and weight, including ranges of each thereof, are approximations that typically may be varied (+) or (−) by increments of 0.1, 1.0, or 10.0, as appropriate. All numerical designations may be understood as preceded by the term “about”. Reagents described herein are exemplary and equivalents of such may be known in the art.

The singular form “a”, “an”, and “the” includes plural references unless the context clearly dictates otherwise.

The term “comprising” means any recited elements are necessarily included and other elements may optionally be included. “Consisting essentially of” means any recited elements are necessarily included, elements that would materially affect the basic and novel characteristics of the listed elements are excluded, and other elements may optionally be included. “Consisting of” means that all elements other than those listed are excluded. Embodiments defined by each of these terms are within the scope of this invention.

Certain terms related to Formula I are defined below.

“Acyl” refers to —CO-alkyl, wherein alkyl is as defined here.

“Aroyl” refers to —CO-aryl, wherein aryl is as defined here.

“Alkoxy” refers to —O-alkyl, wherein alkyl is as defined here.

“Alkenyl” refers to a linear monovalent hydrocarbon radical or a branched monovalent hydrocarbon radical having the number of carbon atoms indicated in the prefix and containing at least one double bond, but no more than three double bonds. For example, (C₂-C₆)alkenyl includes, ethenyl, propenyl, 1,3-butadienyl and the like. Alkenyl can be optionally substituted with substituents, including for example, deuterium (“D”), hydroxyl, amino, mono or di(C₁-C₆)alkyl amino, halo, C₂-C₆ alkenyl ether, cyano, nitro, ethynyl, C₁-C₆ alkoxy, C₁-C₆ alkylthio, —COOH, —CONH₂, mono- or di(C₁-C₆)alkylcarboxamido, —SO₂NH₂, —OSO₂—(C₁-C₆)alkyl, mono or di(C₁-C₆) alkylsulfonamido, aryl, heteroaryl, alkyl or heteroalkylsulfonyloxy, and aryl or heteroarylsulfonyloxy.

“Alkyl” refers to a linear saturated monovalent hydrocarbon radical or a branched saturated monovalent hydrocarbon radical having the number of carbon atoms indicated in the prefix. (C₁-C₆)alkyl can be optionally substituted with substituents, including for example, deuterium (“D”), hydroxyl, amino, mono or di(C₁-C₆) alkyl amino, halo, C₂-C₆ alkenyl ether, cyano, nitro, ethenyl, ethynyl, C₁-C₆ alkoxy, C₁-C₆ alkylthio, —COOH, —CONH₂, mono- or di(C₁-C₆)alkylcarboxamido, —SO₂NH₂, —OSO₂—(C₁-C₆)alkyl, mono or di(C₁-C₆) alkylsulfonamido, aryl, heteroaryl, alkylsulfonyloxy, heteroalkylsulfonyloxy, arylsulfonyloxy or heteroarylsulfonyloxy.

The prefixes (C₁-C_{qq}), C_{1-qq}, and C₁-C_{qq}, wherein qq is an integer from 2-20, have the same meaning. For example, (C₁-C₆)alkyl, C₁₋₆ alkyl, or C₁-C₆ alkyl includes methyl, ethyl, n-propyl, 2-propyl, n-butyl, 2-butyl, tert-butyl, pentyl, and the like. For each of the definitions herein (e.g., alkyl, alkenyl, alkoxy, etc.), when a prefix is not included to indicate the number of main chain carbon atoms in an alkyl portion, the radical or portion thereof will have six or fewer main chain carbon atoms.

“Alkylamino” or mono-alkylamino refers to —NH-alkyl, wherein alkyl is as defined here.

“Alkynyl” refers to a linear monovalent hydrocarbon radical or a branched monovalent hydrocarbon radical having the number of carbon atoms indicated in the prefix and containing at least one triple bond, but no more than two triple bonds. For example, (C₂-C₆)alkynyl includes, ethynyl, propynyl, and the like. Alkynyl can be optionally substituted with substituents, including for example, deuterium (“D”), hydroxyl, amino, mono or di(C₁-C₆)alkyl amino, halo, C₂-C₆ alkenyl ether, cyano, nitro, ethenyl, C₁-C₆ alkoxy, C₁-C₆ alkylthio, —COOH, —CONH₂, mono- or di(C₁-C₆)alkylcarboxamido, —SO₂NH₂, —OSO₂—(C₁-C₆)alkyl, mono or di(C₁-C₆)alkylsulfonamido, aryl, heteroaryl, alkyl or heteroalkylsulfonyloxy, and aryl or heteroarylsulfonyloxy.

“Aryl” refers to a monovalent monocyclic or bicyclic aromatic hydrocarbon radical of 6 to 10 ring atoms which is substituted independently with one to eight substituents, e.g. one, two, three, four of five substituents selected from deuterium (“D”), alkyl, cycloalkyl, cycloalkylalkyl, halo, nitro, cyano, hydroxyl, alkoxy, amino, acylamino, mono-alkylamino, di-alkylamino, haloalkyl, haloalkoxy, heteroalkyl, COR (where R is hydrogen, alkyl, cycloalkyl, cycloalkyl-alkyl, phenyl or phenylalkyl), —(CR′R″)ₙ—COOR (where n is an integer from 0 to 5, R′ and R″ are independently hydrogen or alkyl, and R is hydrogen, alkyl, cycloalkyl, cycloalkylalkyl, phenyl or phenylalkyl) or —(CR′R″)ₙ—CONR^{x}R^{y} (where n is an integer from 0 to 5, R′ and R″ are independently hydrogen or alkyl, and R^{x} and R^{y} are independently selected from hydrogen, alkyl, cycloalkyl, cycloalkylalkyl, phenyl or phenylalkyl). In one embodiment, R^{x} and R^{Y} together is cycloalkyl or heterocyclyl. More specifically the term aryl includes, but is not limited to, phenyl, biphenyl, 1-naphthyl, and 2-naphthyl, and the substituted forms thereof.

“Cycloalkyl” refers to a monovalent cyclic hydrocarbon radical of three to seven ring carbons. The cycloalkyl group can have one or more double bonds and can also be optionally substituted independently with one, two, three or four substituents selected from alkyl, optionally substituted phenyl, or —C(O)R^{z} (where R^{z} is hydrogen, alkyl, haloalkyl, amino, mono-alkylamino, di-alkylamino, hydroxyl, alkoxy, or optionally substituted phenyl). More specifically, the term cycloalkyl includes, for example, cyclopropyl, cyclohexyl, cyclohexenyl, phenylcyclohexyl, 4-carboxycyclohexyl, 2-carboxamidocyclohexenyl, 2-dimethylaminocarbonyl-cyclohexyl, and the like.

“Dialkylamino” or di-alkylamino refers to —N(alkyl)₂, wherein alkyl is as defined here.

“Heteroalkyl” refers to an alkyl radical as defined herein with one, two or three substituents independently selected from cyano, —OR^{w}, —NR^{x}R^{y}, and —S(O)ₚR^{z} (where p is an integer from 0 to 2), with the understanding that the point of attachment of the heteroalkyl radical is through a carbon atom of the heteroalkyl radical. R^{w} is hydrogen, alkyl, cycloalkyl, cycloalkyl-alkyl, aryl, aralkyl, alkoxycarbonyl, aryloxycarbonyl, carboxamido, or mono- or di-alkylcarbamoyl. R^{x} is hydrogen, alkyl, cycloalkyl, cycloalkyl-alkyl, aryl or araalkyl. R^{y} is hydrogen, alkyl, cycloalkyl, cycloalkyl-alkyl, aryl, araalkyl, alkoxycarbonyl, aryloxycarbonyl, carboxamido, mono- or di-alkylcarbamoyl or alkylsulfonyl. R^{z} is hydrogen (provided that p is 0), alkyl, cycloalkyl, cycloalkyl-alkyl, aryl, araalkyl, amino, mono-alkylamino, di-alkylamino, or hydroxyalkyl. Representative examples include, for example, 2-hydroxyethyl, 2,3-dihydroxypropyl, 2-methoxyethyl, benzyloxymethyl, 2-cyanoethyl, and 2-methylsulfonyl-ethyl. For each of the above, R^{w}, R^{x}, R^{y}, and R^{z} can be further substituted by amino, halo, fluoro, alkylamino, di-alkylamino, OH or alkoxy. Additionally, the prefix indicating the number of carbon atoms (e.g., C₁-C₁₀) refers to the total number of carbon atoms in the portion of the heteroalkyl group exclusive of the cyano, —OR^{w}, —NR^{x}R^{y}, or —S(O)ₚR^{z} portions. In one embodiment, R^{x} and R^{y} together is cycloalkyl or heterocyclyl.

“Heteroaryl” refers to a monovalent monocyclic, bicyclic or tricyclic radical of 5 to 12 ring atoms having at least one aromatic ring containing one, two, or three ring heteroatoms selected from N, O, or S, the remaining ring atoms being C, with the understanding that the attachment point of the heteroaryl radical will be on an aromatic ring. The heteroaryl ring is optionally substituted independently with one to eight substituents, preferably one, two, three or four substituents, selected from alkyl, cycloalkyl, cycloalkyl-alkyl, halo, nitro, cyano, hydroxyl, alkoxy, amino, acylamino, mono-alkylamino, di-alkylamino, haloalkyl, haloalkoxy, heteroalkyl, —COR (where R is hydrogen, alkyl, phenyl or phenylalkyl, —(CR′R″)ₙ—COOR (where n is an integer from 0 to 5, R′ and R″ are independently hydrogen or alkyl, and R is hydrogen, alkyl, cycloalkyl, cycloalkyl-alkyl, phenyl or phenylalkyl), or —(CR′R″)ₙ—CONR^{x}R^{y} (where n is an integer from 0 to 5, R′ and R″ are independently hydrogen or alkyl, and R^{x} and R^{y} are, independently of each other, hydrogen, alkyl, cycloalkyl, cycloalkyl-alkyl, phenyl or phenylalkyl). In one embodiment, R^{x} and R^{y} together is cycloalkyl or heterocyclyl. More specifically the term heteroaryl includes, but is not limited to, pyridyl, furanyl, thienyl, thiazolyl, isothiazolyl, triazolyl, imidazolyl, isoxazolyl, pyrrolyl, pyrazolyl, pyridazinyl, pyrimidinyl, benzofuranyl, tetrahydrobenzofuranyl, isobenzofuranyl, benzothiazolyl, benzoisothiazolyl, benzotriazolyl, indolyl, isoindolyl, benzoxazolyl, quinolyl, tetrahydroquinolinyl, isoquinolyl, benzimidazolyl, benzisoxazolyl, benzothienyl, indazolyl, pyrrolopyrymidinyl, indolizinyl, pyrazolopyridinyl, triazolopyridinyl, pyrazolopyrimidinyl, triazolopyrimidinyl, pyrrolotriazinyl, pyrazolotriazinyl, triazolotriazinyl, pyrazolotetrazinyl, hexaaza-indenly, and heptaaza-indenyl and the derivatives thereof. Unless indicated otherwise, the arrangement of the hetero atoms within the ring can be any arrangement allowed by the bonding characteristics of the constituent ring atoms.

“Heterocyclyl” or “cycloheteroalkyl” refers to a saturated or unsaturated non-aromatic cyclic radical of 3 to 8 ring atoms in which one to four ring atoms are heteroatoms selected from O, NR (where R is hydrogen, alkyl, cycloalkyl, cycloalkylalkyl, phenyl or phenylalkyl), P(═O)OR^{w}, or S(O)ₚ (where p is an integer from 0 to 2), the remaining ring atoms being C, wherein one or two C atoms can optionally be replaced by a carbonyl group. The heterocyclyl ring can be optionally substituted independently with one, two, three or four substituents selected from alkyl, aryl, arylalkyl, heteroaryl, heteroarylalkyl, cycloalkyl, cycloalkylalkyl, halo, nitro, cyano, hydroxyl, alkoxy, amino, mono-alkylamino, di-alkylamino, haloalkyl, haloalkoxy, —COR (where R is hydrogen, alkyl, cycloalkyl, cycloalkylalkyl, phenyl or phenylalkyl), —(CR′R″)ₙ—COOR (n is an integer from 0 to 5, R′ and R″ are independently hydrogen or alkyl, and R is hydrogen, alkyl, cycloalkyl, cycloalkylalkyl, phenyl or phenylalkyl), or —(CR′R″)ₙ—CONR^{x}R^{y} (where n is an integer from 0 to 5, R′ and R″ are independently hydrogen or alkyl, R^{x} and R^{y} are, independently of each other, hydrogen, alkyl, cycloalkyl, cycloalkylalkyl, phenyl or phenylalkyl). More specifically the term heterocyclyl includes, but is not limited to, tetrahydropyranyl, N-methylpiperidin-3-yl, N-methylpyrrolidin-3-yl, 2-pyrrolidon-1-yl, pyrrolidinyl, piperidinyl, morpholinyl, tetrahydrofuranyl, tetrahydrothiofuranyl, 1,1-dioxo-hexahydro-1Δ⁶-thiopyran-4-yl, tetrahydroimidazo[4,5-c]pyridinyl, imidazolinyl, piperazinyl, and piperidin-2-yl and the derivatives thereof. The prefix indicating the number of carbon atoms (e.g., C₃-C₁₀ refers to the total number of carbon atoms in the portion of the cycloheteroalkyl or heterocyclyl group exclusive of the number of heteroatoms.

“Heteroacyl” refers to —CO-heteroalkyl, wherein heteroalkyl is as defined here.

“Heteroaroyl” refers to —CO-heteroayl, wherein heteroaryl is as defined here.

“Rₛᵤₗ sulfonyloxy” refers to Rₛᵤₗ—S(═O)₂—O— and includes alkylsulfonyloxy, heteroakylsulfonyloxy, cycloalkylsulfonyloxy, heterocyclylsulfonyloxy, arylsulfonyloxy and heteroarylsulfonyloxy wherein Rₛᵤₗ is alkyl, heteroakyl, cycloalkyl, heterocyclyl, aryl and heteroaryl respectively, and wherein alkyl, heteroakyl, cycloalkyl, heterocyclyl, aryl and heteroaryl are as defined here. Examples of alkylsulfonyloxy include Me-S(═O)₂—O—, Et-S(═O)₂—O—, CF₃—S(═O)₂—O— and the like, and examples of arylsulfonyloxy include:

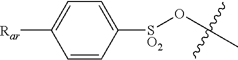

wherein Rₐᵣ is H, methyl, or bromo.

“Substituents” refer to, along with substituents particularly described in the definition of each of the groups above, those selected from: deuterieum, -halogen, —OR′, —NR′R″, —SR′, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NH—C(NH₂)═NH, —NR′C(NH)═NH, —NH—C(NH₂)═NR′, —S(O)R′, —S(O)₂R′, —S(O)₂NR′R″, —NR′S(O)₂R″, —CN, —NO₂, —R′, —N₃, perfluoro(C₁-C₄)alkoxy, and perfluoro(C₁-C₄)alkyl, in a number ranging from zero to the total number of open valences on the radical; and where R′, R″ and R″ are independently selected from hydrogen, C₁₋₈ alkyl, C₃₋₆ cycloalkyl, C₂₋₈ alkenyl, C₂₋₈ alkynyl, unsubstituted aryl and heteroaryl, (unsubstituted aryl)-C₁₋₄ alkyl, and unsubstituted aryloxy-C₁₋₄ alkyl, aryl substituted with 1-3 halogens, unsubstituted C₁₋₈ alkyl, C₁₋₈alkoxy or C₁₋₈ thioalkoxy groups, or unsubstituted aryl-C₁₋₄ alkyl groups. When R′ and R″ are attached to the same nitrogen atom, they can be combined with the nitrogen atom to form a 3-, 4-, 5-, 6-, or 7-membered ring. For example, —NR′R″ is meant to include 1-pyrrolidinyl and 4-morpholinyl. Other suitable substituents include each of the above aryl substituents attached to a ring atom by an alkylene tether of from 1-4 carbon atoms. Two of the substituents on adjacent atoms of the aryl or heteroaryl ring may optionally be replaced with a substituent of the formula -T²-C(O)(CH₂)_{q}—U³—, wherein T² and U³ are independently —NH—, —O—, —CH₂— or a single bond, and q is an integer of from 0 to 2. Alternatively, two of the substituents on adjacent atoms of the aryl or heteroaryl ring may optionally be replaced with a substituent of the formula -A-(CH₂₎ᵣ—B—, wherein A and B are independently —CH₂—, —O—, —NH—, —S—, —S(O)—, —S(O)₂—, —S(O)₂NR′— or a single bond, and r is an integer of from 1 to 3. One of the single bonds of the new ring so formed may optionally be replaced with a double bond. Alternatively, two of the substituents on adjacent atoms of the aryl or heteroaryl ring may optionally be replaced with a substituent of the formula —(CH₂)ₛ—X⁵—(CH₂)ₜ—, wherein s and t are independently integers of from 0 to 3, and X⁵ is —O—, —NR′—, —S—, —S(O)—, —S(O)₂—, or —S(O)₂NR′—. The substituent R′ in —NR′— and —S(O)₂NR′— is selected from hydrogen or unsubstituted C₁₋₆ alkyl.

Certain compounds utilized in the present invention possess asymmetric carbon atoms (optical centers) or double bonds; the racemates, diastereomers, geometric isomers, regioisomers and individual isomers (e.g., separate enantiomers) are all intended to be encompassed within the scope of the present invention. The compounds of the present invention may also contain unnatural proportions of atomic isotopes at one or more of the atoms that constitute such compounds. For example, the compounds may be radiolabeled with radioactive isotopes, such as for example, and without limitation, tritium (³H), iodine-125 (¹²⁵I) or carbon-14 (¹⁴C). All isotopic variations of the compounds of the present invention, whether radioactive or not, are intended to be encompassed within the scope of the present invention.

Other terms related to this invention are defined below.

“Administering” or “administration of” a drug to a patient (and grammatical equivalents of this phrase) refers to direct administration, which may be administration to a patient by a medical professional or may be self-administration, and/or indirect administration, which may be the act of prescribing a drug. For example, a physician who instructs a patient to self-administer a drug and/or provides a patient with a prescription for a drug is administering the drug to the patient.

“Cancer” refers to malignant solid tumors of potentially unlimited growth, as well as various blood cancers that may originate from cancer stem cells in the bone marrow, which can expand locally by invasion and systemically by metastasis. Examples of cancers include, but are not limited to, cancer of the adrenal gland, bone, brain, breast, bronchi, colon and/or rectum, gallbladder, gastrointestinal tract, head and neck, kidneys, larynx, liver, lung, neural tissue, pancreas, prostate, parathyroid, skin, stomach, and thyroid. Other examples of cancers include, adenocarcinoma, adenoma, basal cell carcinoma, cervical dysplasia and in situ carcinoma, Ewing's sarcoma, epidermoid carcinomas, giant cell tumor, glioblastoma multiforma, hairy-cell tumor, intestinal ganglioneuroma, hyperplastic corneal nerve tumor, islet cell carcinoma, Kaposi's sarcoma, leiomyoma, leukemias, lymphomas, malignant carcinoid, malignant melanomas, malignant hypercalcemia, marfanoid habitus tumor, medullary carcinoma, metastatic skin carcinoma, mucosal neuroma, myelodisplastic syndrome, myeloma, mycosis fungoides, neuroblastoma, osteosarcoma, osteogenic and other sarcoma, ovarian tumor, pheochromocytoma, polycythermia vera, primary brain tumor, small-cell lung tumor, squamous cell carcinoma of both ulcerating and papillary type, seminoma, soft tissue sarcoma, retinoblastoma, rhabdomyosarcoma, renal cell tumor or renal cell carcinoma, veticulum cell sarcoma, and Wilm's tumor. Examples of cancers also include astrocytoma, a gastrointestinal stromal tumor (GIST), a glioma or glioblastoma, renal cell carcinoma (RCC), hepatocellular carcinoma (HCC), and pancreatic neuroendocrine cancer.

“Combination therapy” or “combination treatment” refers to the use of two or more drugs in therapy, i.e., use of a hypoxia activated prodrug as described herein together with one or more HDR inhibitors, and optionally other anti cancer agent(s), to treat cancer. Administration in “combination” refers to the administration of two or more agents (e.g., a hypoxia activated prodrug and an HDR inhibitor, and optionally one or more anti cancer agents, for treating cancer) in any manner in which the pharmacological effects of both are manifest in the patient at the same time. Thus, administration in combination does not require that a single pharmaceutical composition, the same dosage form, or the same route of administration be used for administration of both agents or that the two agents be administered at precisely the same time. For example, and without limitation, it is contemplated that an HDR inhibitor can be administered with a hypoxia activated prodrug in accordance with the present invention as a combination therapy.

“Hyperproliferative disease” refers to a disease characterized by cellular hyperproliferation (e.g., an abnormally increased rate or amount of cellular proliferation). Cancer is a hyperproliferative disease. Examples of hyperproliferative diseases other than cancer include, but are not limited to, allergic angiitis and granulomatosis (Churg-Strauss disease), asbestosis, asthma, atrophic gastritis, benign prostatic hyperplasia, bullous pemphigoid, coeliac disease, chronic bronchitis and chronic obstructive airway disease, chronic sinusitis, Crohn's disease, demyelinating neuropathies, dermatomyositis, eczema including atopic dermatitis, eustachean tube diseases, giant cell arteritis, graft rejection, hypersensitivity pneumonitis, hypersensitivity vasculitis (Henoch-Schonlein purpura), irritant dermatitis, inflammatory hemolytic anemia, inflammatory neutropenia, inflammatory bowel disease, Kawasaki's disease, multiple sclerosis, myocarditis, myositis, nasal polyps, nasolacrimal duct diseases, neoplastic vasculitis, pancreatitis, pemphigus vulgaris, primary glomerulonephritis, psoriasis, periodontal disease, polycystic kidney disease, polyarteritis nodosa, polyangitis overlap syndrome, primary sclerosing cholangitis, rheumatoid arthritis, serum sickness, surgical adhesions, stenosis or restenosis, scleritis, scleroderma, strictures of bile ducts, strictures (of duodenum, small bowel, and colon), silicosis and other forms of pneumoconiosis, type I diabetes, ulcerative colitis, ulcerative proctitis, vasculitis associated with connective tissue disorders, vasculitis associated with congenital deficiencies of the complement system, vasculitis of the central nervous system, and Wegener's granulomatosis.

“Hypoxia activated prodrug” refers to a drug that is less active or inactive under normoxia than under hypoxia or anoxia. Hypoxia activated prodrugs include drugs that are activated by a variety of reducing agents and reducing enzymes, including without limitation single electron transferring enzymes (such as cytochrome P450 reductases) and two electron transferring (or hydride transferring) enzymes (see U.S. Pat. App. Pub. Nos. 2005/0256191, 2007/0032455, and 2009/0136521, and PCT Pub. Nos. 2000/064864, 2004/087075, and 2007/002931, each of which is incorporated herein by reference). The hypoxia activated prodrugs useful in the methods of the present invention include compounds of Formula I, including but not limited to compounds where Z₃, as defined by that formula, is a 2-nitroimidazole moiety, as well as other hypoxia activated prodrugs that induce DNA damage repairable by the HDR system. Examples of particular hypoxia activated prodrugs useful in the methods of the invention include without limitation TH-281, TH-302, and TH-308. Methods of synthesizing, formulating, and using TH-302 and other compounds of Formula I are described in PCT Pub. Nos. 2007/002931, 2008/083101, 2010/048330, 2012/006032, and 2012/009288, and U.S. App. No. 61/475,844, filed 15 Apr. 11, each of which is incorporated herein by reference. Examples of other hypoxia activated prodrugs include without limitation, PR104, AQ4N, CEN-209 (also known as SN30000), and tirapazamine. Methods of synthesizing PR104 are described in US Pat. App. Pub. No 2007/0032455, incorporated herein by reference. Methods for making tirapazamine, CEN-209, and AQ4N are well known to one of skill in the art.

“Patient” or “subject” refers to mammals, particularly humans, and so includes animals of veterinary and research interest, such as simians, cattle, horses, dogs, cats, and rodents with cancer or another hyperproliferative disease.

“Pharmaceutically acceptable” refers to a safe and non-toxic substance that is suitable for administration to a patient according to the present invention.

“Pharmaceutically acceptable salt” refers to pharmaceutically acceptable salts derived from a variety of organic and inorganic counter ions well known in the art that include, when the molecule contains an acidic functionality, by way of example only, sodium, potassium, calcium, magnesium, ammonium, and tetraalkylammonium, and when the molecule contains a basic functionality, salts of organic or inorganic acids, such as hydrochloride, hydrobromide, tartrate, mesylate, acetate, maleate, and oxalate. Suitable salts include those described in P. Heinrich Stahl, Camille G. Wermuth (Eds.), Handbook of Pharmaceutical Salts Properties, Selection, and Use; 2002.

TH302 or TH-302 refers to the compound of formula:

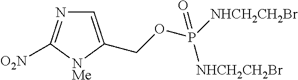

and includes a pharmaceutically acceptable salt thereof. Compounds with identical or similar mechanism of action as that of TH-302, include other hypoxia activated prodrugs capable of alkylating DNA; non limiting example of which include TH-281 and TH-308.

TH281 or TH-281 refers to the compound of formula:

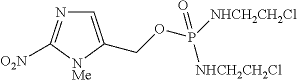

and includes a pharmaceutically acceptable salt thereof.

TH308 or TH-308 refers to the compound of formula:

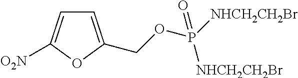

and includes a pharmaceutically acceptable salt thereof.

“Therapeutically effective amount” of a drug or an agent refers to an amount of the drug or the agent that, when administered to a patient with cancer or another hyperproliferative disease, will have the intended therapeutic effect, e.g., alleviation, amelioration, palliation or elimination of one or more manifestations of cancer or another hyperproliferative disease in the patient. A therapeutic effect does not necessarily occur by administration of one dose, and may occur only after administration of a series of doses. Thus, a therapeutically effective amount may be administered in one or more administrations.

“Treating” or “treatment of” a condition or patient refers to taking steps to obtain beneficial or desired results, including clinical results. For purposes of this invention, beneficial or desired clinical results include, but are not limited to, alleviation or amelioration of one or more symptoms of cancer or another hyperproliferative disease including conditional survival and reduction of tumor load or volume; diminishment of extent of disease; delay or slowing of disease progression; amelioration, palliation, or stabilization of the disease state; or other beneficial results.

TH-302 is a hypoxia-activated prodrug whose mechanism of action includes DNA alkylation resulting in DNA cross-links. The present invention arose in part from the discovery that TH-302 activity is potentiated in HDR impaired cells (see Table 8 below), which model cancer cells treated with an HDR inhibitor, whereas inhibition of other DNA repair processes, e.g., an inhibition by a PARP inhibitor, failed to potentiate TH-302 activity (see Tables 2, 4, 6, and 7 below).

Thus, the present invention provides new methods of treating cancer with a compound of Formula I, e.g., TH-302, and other hypoxia activated prodrugs with identical mechanism of action (inducing DNA damage repairable via the HDR system). In some embodiments, such methods comprise administering TH-302 at a dose and frequency known to have anti-tumor effect (as described herein and in the patent applications listed above) in combination with pharmacological agent(s), including FDA and other regulatory authority approved drugs, that down-regulate (or inhibit) HDR. Such agents include but are not limited to proteosome inhibitors (e.g. bortezomib), HDAC inhibitors (e.g. vorinostat, also known as SaHa), and tyrosine kinase inhibitors (e.g. imatinib, gefitinib, and erlotinib).

The present invention thus provides new methods for administering TH-302 to provide an enhanced anticancer activity and/or therapeutic index over currently used methods. Clinical combinations of agents down-regulating homology directed DNA repair as part or all of their mechanism of action are provided by the invention. In one embodiment, the HDR inhibitor is administered first, and then the TH-302 is administered, including, without limitation 2 hours or longer after the administration of the repair inhibitor is complete. In another embodiment the HDR inhibitor and TH-302 are administered simultaneously. In most embodiments, multiple administrations of each drug are employed in a course of therapy. Illustrative administration schedules are described in more detail below.

### Hypoxia Activated Prodrug Administration

In one aspect, the present invention provides a method of treating cancer comprising administering a therapeutically effective amount of a hypoxia activated prodrug of Formula I and a therapeutically effective amount of an HDR inhibitor to a patient in need of such treatment. In one embodiment, the combination therapy is administered to a patient that has been previously treated with an HDR inhibitor or a hypoxia activated prodrug of Formula I, but the cancer is progressing despite the therapy, or the therapy has been discontinued due to cancer progression. In other embodiments, the patient has not been previously treated with any anti-cancer drug. In other embodiments, the patient has been previously treated with an anti-cancer drug other than an HDR inhibitor or a hypoxia activated prodrug of Formula I.

In one embodiment, the hypoxia activated prodrug of Formula I is selected from the group consisting of TH-281, TH-302, and TH-308. In one embodiment, the hypoxia activated prodrug administered is TH-302. In various embodiments, the TH-302 or other hypoxia activated prodrug of Formula I is administered once daily, once every 3 days, weekly, or once every 3 weeks. In one embodiment, the TH-302 or other hypoxia activated prodrug of Formula I is administered parenterally. In another embodiment, the TH-302 or other hypoxia activated prodrug is administered orally (see provisional U.S. patent application Ser. No. 61/475,844, filed 15 Apr. 2011, incorporated herein by reference).

In one embodiment, the hypoxia activated prodrug is TH-302, which is administered in a daily dose of about 120 mg/m² to about 460 mg/m². In some embodiments, the daily dose of TH-302 is administered in a single dose for 5 consecutive days followed by 2 days of no TH-302 administration, i.e., a one week cycle of therapy. Such a 1 week cycle of therapy can be repeated for 1-3 additional cycles, followed by 1-3 weeks of no drug administration, and this treatment regimen may be repeated one or multiple times. The less frequent the administration, the higher the daily doses of the hypoxia activated prodrug of Formula I administered may be.

In one embodiment, TH-302 or another hypoxia activated prodrug is administered once weekly. In one embodiment, the therapeutically effective amount of TH-302 is a once weekly dose of about 480 mg/m²-about 670 mg/m², or, e.g., 575 mg/m². In another embodiment, the therapeutically effective amount of TH-302 is a daily dose of about 240 mg/m² to about 480 mg/m² administered on days 1 and 8 of a 3 week cycle.

In various embodiments, the hypoxia activated prodrug is TH-302, which is administered intravenously over about 30 minutes in doses of about 240 mg/m², about 340 mg/m², about 480 mg/m², or about 575 mg/m² given once weekly or as a 1 week off followed by 3 weeks on therapy schedule or 3 weeks on followed by 1 week off schedule. In various embodiments, the hypoxia activated prodrug is TH-302, which is administered on days 8, 15 and 22, of 28 day cycles. In another embodiment, the hypoxia activated prodrug is TH-302, which is administered on days 8, 15 and 22, of 42 day cycles.

In various embodiments, the hypoxia activated prodrug is TH-302, which is administered intravenously over about 30-about 60 minutes in doses of about 240 mg/m² to about 480 mg/m² given in Q2 week (once every 2 weeks), or in doses of about 240 mg/m² or 340 mg/m² administered on days 1, 8, and 15 of a 4 week cycle. In another embodiment, such a schedule is employed post surgery, e.g. for treating a brain or other cancer.

When an HDR inhibitor is combined with a hypoxia activated prodrug according to the present invention, the HDR inhibitor is contemplated to be administered in amounts and dosing frequencies as disclosed herein below, or in amounts and frequencies apparent to the skilled artisan in view of this disclosure, or in amounts and frequencies approved by the FDA or other regulatory authority for use in the treatment of cancer.

In one embodiment, the patient's cancer treated is a metastatic cancer or a refractory and/or relapsed cancer that is refractory to first, second, or third line treatment. In another embodiment, the treatment is a first, a second, or a third line treatment. As used herein, the phrase “first line” or “second line” or “third line” refers to the order of treatment received by a patient. First line treatment regimens are treatments given first, whereas second or third line treatment are given after the first line therapy or after the second line treatment, respectively. Therefore, first line treatment is the first treatment for a disease or condition. In patients with cancer, primary treatment can be surgery, chemotherapy, radiation therapy, or a combination of these therapies. First line treatment is also referred to those skilled in the art as primary therapy or primary treatment. Typically, a patient is given a subsequent chemotherapy regimen because the patient did not show a positive clinical or only showed a sub-clinical response to the first line therapy, or the first line treatment has stopped.

In another aspect, the treatment methods of the present invention are used for treating hyperproliferative diseases other than cancer.

Methods of preparation and pharmaceutical compositions of hypoxia activated prodrugs, and other methods of treating cancer by administering various hypoxia activated prodrugs of Formula I are described in Duan et al., J. Med. Chem. 2008, 51, 2412-2420, and PCT Pub. Nos. 2007/002931, 2008/083101, 2010/048330, 2012/006032, and 2012/009288, each of which is incorporated herein by reference. Other methods of treating cancers, which may be used in combination with the methods of the present invention, are known to one of skilled in the art, and are described, for example, in the product descriptions found in the 2010 or more current edition of the Physician's Desk Reference, Medical Economics Company, Inc., Oradell, N.J.; Goodman and Gilman's The pharmacological basis of therapeutics, Eds. Hardman et al., McGraw-Hill. New York. (US) 2011, 12th Ed., and in publications of the U.S. Food and Drug Administration and the NCCN Guidelines (National Comprehensive Cancer Network). Such methods can be appropriately modified by one of skill in the art, in view of this disclosure, to practice the treatment methods of the present invention.

In one embodiment, the TH-302 is provided in 100 mg vials, lyophilized, and dissolved in D5W and administered intravenously (i.v.) over approximately 30-60 minutes via an infusion pump. The infusion volume depends on the total dose given (in mg) during the infusion. If less than about 1000 mg is being infused, about 500 mL of D5W are used for infusion. If the total dose is greater than about 1000 mg, about 1000 mL of D5W are used for infusion.

HDR Inhibitors and their Administration

In certain embodiments, bortezomib, a proteasome inhibitor, is useful in accordance with the methods of the present invention. In some embodiments, bortezomib and a compound of Formula I, e.g., TH-302, or another hypoxia activated prodrug are co-administered to treat cancer, and in other embodiments, additional anti-cancer agents such as melphalan and prednisone, are used. For example, bortezomib; a compound of Formula I, e.g., TH-302; melphalan; and prednisone can be co-administered for the treatment of multiple myeloma and mantle cell lymphoma. Melphalan and prednisone are administered following methods well known to the skilled artisan. In such embodiments, bortezomib can be administered at a dose of 1.3 mg/m² administered either as a bolus intravenous injection or as a subcutaneous injection. Bortezomib can be administered for up to 9,6-week cycles. In cycles 1-4, bortezomib can be administered twice weekly (on days 1, 4, 8, 11, 22, 25, 29, and 32). In cycles 5-9, bortezomib can be administered once weekly (on days 1, 8, 22, and 29).

In certain other embodiments, vorinostat, an HDAC inhibitor, is useful in accordance with the methods of the present invention. In some embodiments, vorinostat and a compound of Formula I, e.g., TH-302, or another hypoxia activated prodrug are co-administered to treat cancer. For example, vorinostat and a compound of Formula I, e.g., TH-302, can be co-administered for the treatment of T-cell lymphoma. In such embodiments, vorinostat can be administered at a dose of 400 mg once daily, orally. If the patient is intolerant to therapy, the dose may be reduced to 300 mg once daily orally. If necessary, the dose may be further reduced to 300 mg once daily for 5 consecutive days each week.

In certain other embodiments, imatinib is useful in accordance with the methods of the present invention. In some embodiments, imatinib and a compound of Formula I, e.g., TH-302, or another hypoxia activated prodrug with identical mechanism of action are co-administered. Illustrative cancers that can be treated in accordance with the methods of the invention include Philadelphia chromosome positive chronic myeloid leukemia (Ph+CML) in chronic phase for newly diagnosed adult and pediatric patients; Philadelphia chromosome positive chronic myeloid leukemia (Ph+CML) in blast crisis (BC), accelerated phase (AP), or in chronic phase (CP) after failure of interferon-alpha therapy; relapsed or refractory Philadelphia chromosome positive acute lymphoblastic leukemia (Ph+ALL) in adult patients; myelodysplastic/myeloproliferative diseases (MDS/MPD) associated with PDGFR (platelet-derived growth factor receptor) gene re-arrangements in adult patients; aggressive systemic mastocytosis (ASM) without the D816V c-Kit mutation or with c-Kit mutational status unknown in adult patients; hypereosinophilic syndrome (HES) and/or chronic eosinophilic leukemia (CEL) containing the FIP1L1-PDGFRα fusion kinase (mutational analysis or FISH demonstration of CHIC2 allele deletion) in adult patients and for patients with HES and/or CEL who are FIP1L1-PDGFRα fusion kinase negative or unknown; unresectable, recurrent and/or metastatic dermatofibrosarcoma protuberans (DFSP) in adult patients; Kit (CD117) positive unresectable and/or metastatic malignant gastrointestinal stromal tumors (GIST) in adult patients; and adjuvant treatment following resection of Kit (CD117) positive GIST in adult patients.

When administered in accordance with the present invention, imatinib can be administered in the following amounts: adults with Ph+CML CP, adults with MDS/MPD, adults with metastatic and/or unresectable GIST, adjuvant treatment of adults with GIST, and patients with mild to moderate hepatic impairment: 400 mg/day; adults with ASM and adults with HES/CEL: 100 mg/day or 400 mg/day; adults with Ph+CML AP or BC and adults with Ph+ALL: 600 mg/day; pediatrics with Ph+CML CP: 340 mg/m²/day; adults with DFSP: 800 mg/day; and patients with severe hepatic impairment: 300 mg/day.

Accordingly, imatinib can be administered in a daily amount of from 100 mg to 800 mg together with a hypoxia activated prodrug of Formula I, e.g. TH-302, and optionally another anti cancer agent, for the treatment of cancer, for example of leukemia, such as Ph+CML) in blast crisis (BC), accelerated phase (AP), or in chronic phase (CP), and Ph+ALL, myelodysplastic/myeloproliferative diseases, aggressive systemic mastocytosis, hypereosinophilic syndrome (HES) and/or chronic eosinophilic leukemia, and GIST. In certain embodiments, 400 mg or 600 mg imatinib are administered once daily. In other embodiments, a daily dose of 800 mg imatinib is administered in 400 mg unit dose forms, separately administered in time.

In certain other embodiments, gefitinib is useful in accordance with the methods of the present invention. In some embodiments, it is co-administered with a compound of Formula I, e.g., TH-302, or another hypoxia activated prodrug of identical mechanism of action for treating locally advanced or metastatic non-small cell lung cancer (NSCLC) with activating mutations of EGFR-TK. In some embodiments, gefitinib is administered once daily, orally, in an amount of 250 mg.

In certain other embodiments, erlotinib is useful in accordance with the methods of the present invention. In some embodiments, it is co-administered with a compound of Formula I, e.g., TH-302, or another hypoxia activated prodrug of identical mechanism of action, and in other embodiments, additional anti-cancer agents are administered as well, for the following: maintenance treatment of patients with locally advanced or metastatic non-small cell lung cancer whose disease has not progressed after four cycles of platinum-based first-line chemotherapy; treatment of locally advanced or metastatic non-small cell lung cancer after failure of at least one prior chemotherapy regimen; and first-line treatment of patients with locally advanced, unresectable or metastatic pancreatic cancer, e.g., in combination with gemcitabine. In some embodiment, the dose of erlotinib for treating non-small cell lung cancer according to the present invention is 150 mg/day. In some other embodiments, the dose of erlotinib for treating for treating pancreatic cancer according to the present invention is 100 mg/day.

Other methods in accordance with the present invention can be practiced by adapting, in accordance with the teachings herein, the methods and compounds described in the following references, each of which is incorporated herein by reference: Choudhury et al., “Targeting homologous recombination using imatinib results in enhanced tumor cell chemosensitivity and radiosensitivity,” Mol. Cancer Ther., 2009, 8(1):203-213; Evers B, et al., “Targeting homologous recombination repair defects in cancer,” Trends Pharmacol. Sci., 2010, 31(8):372-380; Helleday, “Homologous recombination in cancer development, treatment and development of drug resistance,” Carcinogenesis, 2010, 31(6): 955-960; Li et al., “Erlotinib attenuates homologous recombinational repair of chromosomal breaks in human breast cancer cells,” Cancer Res. 2008, 68(22):9141-9146; Singh et al., “Suberoylanilide hydroxyamic acid modification of chromatin architecture affects DNA break formation and repair.” Int. J. Radiat. Oncol. Biol. Phys., 2010, 76(2):566-573; Tanaka et al., “Gefitinib radiosensitizes non-small cell lung cancer cells by suppressing cellular DNA repair capacity,” Clin. Cancer Res., 2008, 14(4):1266-1273; and Yarde et al., “Targeting the Fanconi anemia/BRCA pathway circumvents drug resistance in multiple myeloma,” Cancer Res., 2009, 69(24):9367-9375.

The invention also provides methods for determining whether a cancer is likely to be susceptible or resistant to treatment with TH-302 and compounds with identical mechanism of action. In these methods, cancer cells from a patient are assessed to determine the level of HDR activity in them. In brief, the lower the HDR activity in the cell, the more susceptible the cancer to TH-302 (and vice-versa). Any known method for assessing HDR activity may be used in the method.

### EXAMPLES

TH-302, a hypoxia-activated prodrug currently in clinical trials for the treatment of cancer, releases the DNA cross-linking bromo-isophosphoramidate mustard (Br-IPM) under hypoxic conditions and exhibits about 400-fold hypoxia-enhanced cytotoxicity in multiple human cancer cell lines in vitro. In these examples, pharmacological tools were utilized to test the underlying mechanisms of DNA repair processes involved in the cellular response to TH-302. In vitro cytotoxicity assays were used as the primary read-out.

### Example 1

### Effect of a PARP Inhibitor on TH-302

The effect of the PARP inhibitor, ABT-888, on TH-302 activity was investigated in three human cancer cell lines: human non small cell lung H460, human melanoma A375, and human colorectal carcinoma HCT116.

Cells were pretreated with ABT-888 for 1 h under normoxia, and then co-incubated with TH-302 for additional 2 h under either normoxia or hypoxia. After 3 days of incubation in the presence of ABT-888, cell viability was determined using AlamarBlue. For the temozolomide (TMZ) group, cells were co-treated with temozolomide and ABT-888 for 3 days after 1 h pretreatment with ABT-888. The results are tabulated in Tables 1-6 below.

The results demonstrated that TH-302 activity was not substantially affected by the presence of ABT-888 (see, Tables 2, 4, and 6). In contrast, the activity of the monoalkylating agent temozolomide was enhanced by ABT-888 in these cancer cell lines (see, Tables 1, 3, and 5). Similarly, the sensitivity of EM9 cells (deficient in base excision repair gene XRCC1) exhibited a heightened sensitivity to temozolomide but not to TH-302 (Table 7).

Taken together, these results indicate that single-strand break (SSB) DNA repair mechanisms are not involved or are insubstantially involved in the repair of TH-302 lesions. The role of homology-directed DNA repair (HDR) on TH-302 was investigated in wild type and HDR deficient chinese hamster ovary (CHO) cells. The results show that lines deficient in homologous recombination directed repair exhibited marked sensitivity to TH-302 than the parental control line. TH-302 activity can be modulated by pharmacological modulators of both reductases and DNA damage and repair pathways. TH-302 activity can be inhibited by the flavin reductase inhibitor DPI. TH-302 activity was not affected by the PARP inhibitor ABT-888. These results support that HDR DNA repair mechanisms contribute to TH-302's anti cancer cytotoxicity.

TABLE 1
(H460 cell lines)

| | IC₅₀ (μM); |
|---|---|
| Compound | Normoxia |
| Temozolomide (TMZ) | 540 |
| TMZ + 1 μM ABT888 | 85 |
| TMZ + 10 μM ABT888 | 21 |

TABLE 2
(H460 cell lines)

IC₅₀ (μM)

| Compound | Normoxia | Hypoxia |
|---|---|---|
| TH302 | 47 | 0.2 |
| TH302 + 1 μM ABT888 | 39 | 0.1 |
| TH302 + 10 μM ABT888 | 36 | 0.2 |

TABLE 3
(HCT116 cell lines)

| | IC₅₀ (μM); |
|---|---|
| Compound | Normoxia |
| TMZ | 890 |
| TMZ + 0.5 μM ABT-888 | 350 |
| TMZ + 5 μM ABT-888 | 140 |

TABLE 4
(HCT116 cell lines)

IC₅₀ (μM)

| Compound | Normoxia | Hypoxia |
|---|---|---|
| TH302 | 61 | 0.1 |
| TH302 + 0.5 μM ABT888 | 84 | 0.1 |
| TH302 + 5 μM ABT888 | 85 | 0.2 |

TABLE 5
(A375 cell lines)

| | IC₅₀ (μM); |
|---|---|
| Compound | Normoxia |
| TMZ | 510 |
| TMZ + 0.5 μM ABT-888 | 410 |
| TMZ + 5 μM ABT-888 | 210 |

TABLE 6
(A375 cell lines)

IC₅₀ (μM)

| Compound | Normoxia | Hypoxia |
|---|---|---|
| TH302 | 230 | 1.5 |
| TH302 + 0.5 μM ABT888 | 230 | 2.0 |
| TH302 + 5 μM ABT888 | 210 | 2.0 |

TABLE 7

| | | | Fold | | | Fold |
|---|---|---|---|---|---|---|
| | IC₅₀ (μM) | | difference | IC₅₀ (μM) | | difference |
| | Normoxia | | in | Anoxia | | in |
| Compound | AA8 | EM9 | sensitivity | AA8 | EM9 | sensitivity |
| TH-302 | >1000 | 760 | >1.3 | 10 | 14 | 1 |
| Tirapazamine | >1000 | >1000 | | 50 | 15 | 3 |
| Temozolomide | >1000 | 168 | >5.9 | | | |

### Example 2

### TH-302 is Active in HDR Impaired Cells

The wild type AA8 cells and the HDR impaired irs1SF and UV41 cells were treated with TH-302 for 2 h, washed, and then incubated for 3 days. At the end of the incubation, viable cells were quantified using alamar blue. TH-302's hypoxia selective, anti cancer activity was observed in homology-directed DNA repair (HDR) deficient cell line UV41 and irs1SF. The results indicate that HDR can be involved in the DNA repair process initiated by TH-302. The results demonstrate that the combination of TH-302 with agents that inhibit repair of TH-302 induced DNA damage, especially HDR, is contemplated to result in enhanced efficacy of this clinical stage anti-cancer agent.

TABLE 8

| | IC₅₀ (μM) | | |
|---|---|---|---|
| Cell line | Air | N₂ | HCR |
| AA8 | 340 | 1.1 | 310 |
| irs1SF | 95 | 0.5 | 190 |
| UV41 | 16 | 0.02 | 800 |

While certain embodiments have been illustrated and described in the foregoing examples, it will be understood that changes and modifications can be made in the foregoing processes in accordance with ordinary skill in the art without departing from the present invention in its broader aspects as defined in the following claim.

It should be understood that although the present invention has been specifically disclosed by certain aspects, embodiments, and optional features, modification, improvement and variation of such aspects, embodiments, and optional features can be resorted to by those skilled in the art, and that such modifications, improvements and variations are considered to be within the scope of this disclosure.

The inventions have been described broadly and generically herein. Each of the narrower species and subgeneric groupings falling within the generic disclosure also form part of the invention. In addition, where features or aspects of the invention are described in terms of Markush groups, those skilled in the art will recognize that the invention is also thereby described in terms of any individual member or subgroup of members of the Markush group.

1. A method of treating cancer comprising administering a therapeutically effective amount of a compound of Formula I: 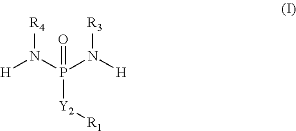
wherein
Y₂ is O, S, NR₆, NCOR₆, or NSO₂R₆
R₆ is C₁-C₆ alkyl, C₁-C₆ heteroalkyl, aryl, or heteroaryl;
R₃ and R₄ are independently selected from the group consisting of 2-haloalkyl, 2-alkylsulfonyloxyalkyl, 2-heteroalkylsulfonyloxyalkyl, 2-arylsulfonyloxyalkyl, and 2-heteroalkylsulfonyloxyalkyl;
R₁ has the formula L-Z₃;
L is C(Z₁)₂;
each Z₁ independently is hydrogen, halogen, C₁-C₆ alkyl, C₁-C₆ heteroalkyl, aryl, heteroaryl, C₃-C₈ cycloalkyl, heterocyclyl, C₁-C₆ acyl, C₁-C₆ heteroacyl, aroyl, or heteroaroyl;
or L is: 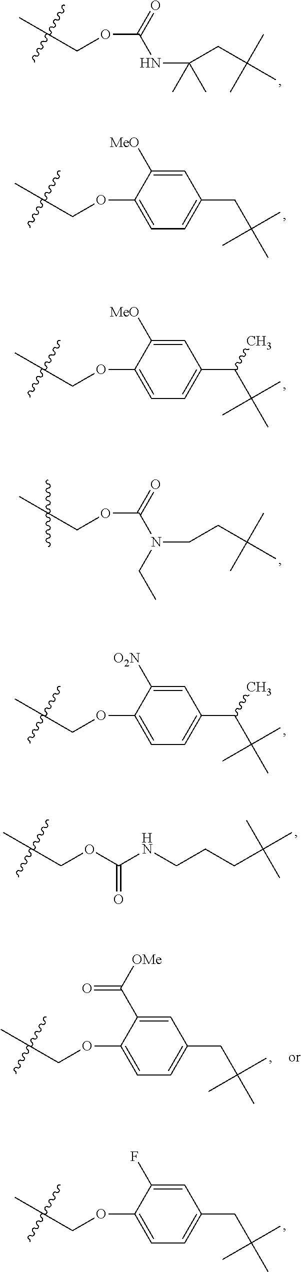
Z₃ is a bioreductive group having a formula selected from the group consisting of: 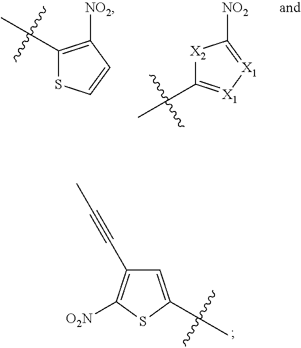
each X₁ is independently N or CR₈;
X₂ is NR₇, S, or O;
each R₇ is independently C₁-C₆ alkyl, C₁-C₆ heteroalkyl, C₃-C₈ cycloalkyl, heterocyclyl, aryl or heteroaryl;
and R₈ is independently hydrogen, halogen, cyano, CHF₂, CF₃, CO₂H, amino, C₁-C₆ alkyl, C₁-C₆ heteroalkyl, C₁-C₆ cycloalkyl, C₁-C₆ alkoxy, C₁-C₆ alkylamino, C₁-C₆ dialkylamino, aryl, CON(R₇)₂, C₁-C₆ acyl, C₁-C₆ heteroacyl, aroyl or heteroaroyl;
or a pharmaceutically acceptable salt thereof;
in combination with a therapeutically effective amount of a pharmacological agent that down-regulates or inhibits homology directed repair (HDR) of DNA to a patient in need thereof.

2. The method of claim 1, wherein the pharmacological agent that down-regulates or inhibits homology directed repair of DNA is bortezomib, vorinostat, imatinib, gefitinib, or erlotinib.

3. The method of claim 1, wherein the compound of Formula I is TH-302.

4. The method of claim 1, wherein the cancer is a blood cancer, a GIST, a pancreatic cancer, or a lung cancer.

5. A pharmaceutically acceptable formulation comprising a compound of Formula I: 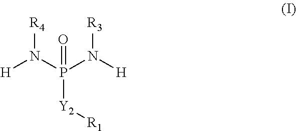
wherein
Y₂ is O, S, NR₆, NCOR₆, or NSO₂R₆
R₆ is C₁-C₆ alkyl, C₁-C₆ heteroalkyl, aryl, or heteroaryl;
R₃ and R₄ are independently selected from the group consisting of 2-haloalkyl, 2-alkylsulfonyloxyalkyl, 2-heteroalkylsulfonyloxyalkyl, 2-arylsulfonyloxyalkyl, and 2-heteroalkylsulfonyloxyalkyl;
R₁ has the formula L-Z₃;
L is C(Z₁)₂;
each Z₁ independently is hydrogen, halogen, C₁-C₆ alkyl, C₁-C₆ heteroalkyl, aryl, heteroaryl, C₃-C₈ cycloalkyl, heterocyclyl, C₁-C₆ acyl, C₁-C₆ heteroacyl, aroyl, or heteroaroyl;
or L is: 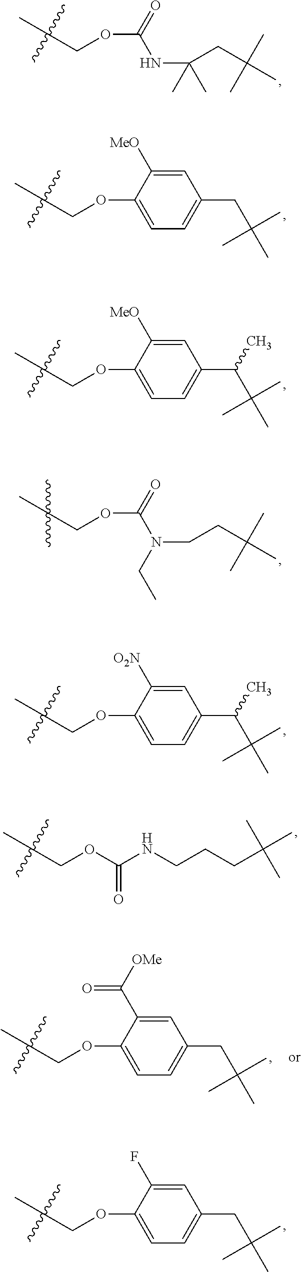
Z₃ is a bioreductive group having a formula selected from the group consisting of: 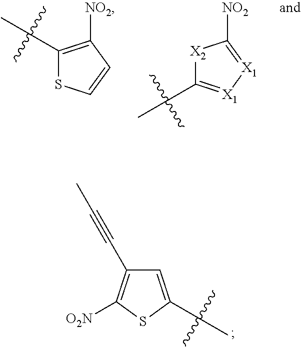
each X₁ is independently N or CR₈;
X₂ is NR₇, S, or O;
each R₇ is independently C₁-C₆ alkyl, C₁-C₆ heteroalkyl, C₃-C₈ cycloalkyl, heterocyclyl, aryl or heteroaryl;
and R₈ is independently hydrogen, halogen, cyano, CHF₂, CF₃, CO₂H, amino, C₁-C₆ alkyl, C₁-C₆ heteroalkyl, C₁-C₆ cycloalkyl, C₁-C₆ alkoxy, C₁-C₆ alkylamino, C₁-C₆ dialkylamino, aryl, CON(R₇)₂, C₁-C₆ acyl, C₁-C₆ heteroacyl, aroyl or heteroaroyl;
or a pharmaceutically acceptable salt thereof;
a pharmacological agent that down-regulates or inhibits homology directed repair of DNA, and at least a pharmaceutically acceptable excipient.

6. The pharmaceutically acceptable formulation of claim 5, wherein the pharmacological agent that down-regulates or inhibits homology directed repair of DNA is bortezomib, vorinostat, imatinib, gefitinib, or erlotinib.

7. The pharmaceutically acceptable formulation of claim 5 wherein the compound of Formula I is TH-302.

8. The pharmaceutically acceptable formulation of claim 5, which contains a therapeutically effective amount of the compound of Formula I.

9. The pharmaceutically acceptable formulation of claim 5, which contains a therapeutically effective amount of the pharmacological agent that down-regulates or inhibits homology directed repair of DNA.

10.-13. (canceled)

14. The method of claim 2, wherein the compound of Formula I is TH-302.

15. The method of claim 14, wherein the cancer is a blood cancer, a GIST, a pancreatic cancer, or a lung cancer.

16. The pharmaceutically acceptable formulation of claim 6, wherein the compound of Formula I is TH-302.

17. The pharmaceutically acceptable formulation of claim 16, which contains a therapeutically effective amount of the compound of Formula I.

18. The pharmaceutically acceptable formulation of claim 17, which contains a therapeutically effective amount of the pharmacological agent that down-regulates or inhibits homology directed repair of DNA.
